(19) United States
(12) Patent Application Publication
(10) Pub. No.: US 2026/0124328 A1
(43) Pub. Date: May 7, 2026

(54) Treatment Transducer for In Vivo Disinfection of Surfaces of Electrically Conductive Medical Devices,Parts and Components

(71) Applicant: Solenic Medical, Inc., Addison, TX (US)

(72) Inventors: John C. Tepper, Carollton, TX (US); Varun Sadaphal, Dallas, TX (US); Bibin Prasad, Dallas, TX (US); Sandy Huang, Addison, TX (US); Andrei Mitrofan, Addison, TX (US); Jason Fisher, Addison, TX (US)

(73) Assignee: Solenic Medical, Inc., Addison, TX (US)

(21) Appl. No.: 19/344,078

(22) Filed: Sep. 29, 2025

Related U.S. Application Data
(63) Continuation-in-part of application No. 18/934,339, filed on Nov. 1, 2024.

(51) Int. Cl. A61L 2/04; A61F 2/30; A61L 103/15; A61N 1/40

(52) U.S. Cl. CPC A61L 2/04; A61F 2/30; A61N 1/403; A61F 2002/30668; A61F 2002/30719; A61F 2007/009; A61L 2103/15; A61L 2202/14

(57) ABSTRACT

A treatment transducer for a generator of alternating magnetic fields for in vivo disinfection of electrically conductive surfaces of medical implants, which may be used with a wide variety of implants and medical devices. The treatment head includes an enclosure for enveloping a portion of a body in which an implant is to be treated, and which contains a plurality of transducers for converting a radio-frequency signal to generating a shaped alternating magnetic field.

### FIELD OF THE INVENTION

This application claims benefit of the filing date of U.S. Non-Provisional Utility patent application Ser. No. 18/934,339, our docket FGP23SLC2NP, filed on Nov. 1, 2024, by John C. Tepper et al., which claims benefit of the filing date of U.S. Provisional Patent Application 63/595,261, our docket FGP23SLC2, filed on Nov. 11, 2023, by John C. Tepper. This invention is related to systems and methods for disinfecting metallic surfaces of electrically conductive medical devices, parts and components which have been implanted internally to patients, including but not limited to metallic implants and carbon fiber implants.

### INCORPORATION BY REFERENCE

The following patent applications and patents are incorporated by reference in their entireties, including drawings:
a. WIPO International Patent Application WO2022/140226 A1, published on Jun. 30, 2022, by inventors David Greenberg, et al.;
b. U.S. Non-Provisional Utility patent application Ser. No. 18/934,339, our docket FGP23SLC2NP, filed on Nov. 1, 2024, by John C. Tepper et al.; and
c. U.S. Provisional Patent Application 63/595,261, our docket FGP 23SLC2, filed on Nov. 11, 2023, by John C. Tepper. This invention is related to systems and methods for disinfecting metallic surfaces of electrically conductive medical devices, parts and components which have been implanted internally to patients, including but not limited to metallic implants and carbon fiber implants.

### BACKGROUND OF THE INVENTION

A treatment transducer is disclosed for a generator of alternating magnetic fields for in vivo disinfection of electrically conductive surfaces of medical implants, which may be used with a wide variety of implants and appliances. The treatment transducer includes an enclosure for enveloping a portion of a body in which an implant is to be treated, and which contains a plurality of conductive windings for converting a radio-frequency electrical signal to generating a shaped alternating magnetic field.

### SUMMARY OF THE INVENTION

A treatment transducer is disclosed for a generator of alternating magnetic fields for in vivo disinfection of electrically conductive surfaces of medical implants, which may be used with a wide variety of implants and medical devices. The treatment head includes an enclosure for enveloping a portion of a body in which an implant is to be treated, and which contains a plurality of transducers for converting a radio-frequency signal to generating a shaped alternating magnetic field.

### BRIEF DESCRIPTION OF DRAWINGS

Features and advantages of embodiments of the present invention will become apparent from the appended claims, the following detailed description of one or more example embodiments, and the corresponding figures. Where considered appropriate, reference labels have been repeated among the figures to indicate corresponding or analogous elements. This patent application file contains at least one drawing executed in color. Copies of this patent or patent application publication with color drawing(s) will be provided by the Office upon request and payment of the necessary fee.

FIG. 1 shows a perspective view of a treatment head according to at least one embodiment of the related invention.

FIG. 2 shows a top-down view of a treatment head according to at least one embodiment of the present invention.

FIG. 3 shows a side view of a treatment head according to at least one embodiment of the present invention.

FIG. 4 illustrates an example coil interconnection plan according to at least one embodiment of the present invention.

FIG. 5 provides a block diagram of system components according to at least one embodiment of the present invention.

FIG. 6 depicts an entire example system embodiment of an AMF treatment system according to the present and related inventions.

FIG. 7 shows the general phases of thermal disruption of biofilms on targeted surfaces of a prosthetic joint using AMF.

FIG. 8 illustrates how such an AMF treatment transducer is positioned over and around a prosthetic knee joint.

FIG. 9 shows the general phases of development of a make-and-model specific AMF treatment transducer device according to the present invention.

FIG. 10 shows a perspective view of a treatment head according to at least one embodiment of the present invention for a second specific make-and-model of prosthetic knee.

FIG. 11 shows a side view of the device of FIG. 10.

FIG. 12 shows a side view of the device of FIG. 10.

### DETAILED DESCRIPTION OF THE DISCLOSED EMBODIMENT(S) OF THE INVENTION

Prosthetic Joint Infections (PJIs) are debilitating and increasing in occurrence globally. An average lifetime cost of care has been estimated to be $415,000 per prosthetic joint, per U.S. CMS Coding Data (Q2/24-Q1/25), and there remains an unacceptably high rate of morbidity and mortality.

Per the AcuityMD US Claims Database (Q2-2025 to Q1-2025), the three standards of care for PJIs have unacceptably high failure rates:
a. using antibiotics, 37% failure rate treating prosthetic knees, 37% failure rate treating prosthetic hips, and 47% failure rate treating prosthetic shoulders;
b. using debridement, antibiotics and implant retention (DAIR), 47%, 33%, 22% failure rates treating prosthetic knees, hips and shoulders, respectively; and
c. using two-stage revision, 19%, 19%, 13% failure rates treating prosthetic knees, hips and shoulders, respectively.

An alternative to, or supplement to, antibiotics, DAIR and two-stage revision includes alternating magnetic field (AMF) treatment. AMF is a non-invasive approach to treat infections of implanted medical devices, such as knee or hip implants. Previous patents have disclosed early attempts to provide an external transducer coil which generates a time-varying AMF in the vicinity of a metal implant. The AMF induces surface electrical currents on the implant's electrically conductive metallic portions, which heat the surface of the implant. In the case of an infected implant, bacteria (which may be in the form of a biofilm) adhere to the surface within this heated area; the heat can eradicate pathogens or sensitize them to additional antimicrobial treatments such as antibiotics. One such patent is WO 2022/140226A1 , published on 30 Jun. 2022, by the World Intellectual Property Organization (WIPO) by inventors David Greenberg, et al., for applicant Solenic Medical Inc. of Texas, United States.

Reference will now be made to the drawings wherein like structures may be provided with like suffix reference designations. In order to show the structures of various embodiments more clearly, the drawings included herein are diagrammatic representations of structures. Thus, the actual appearance of the fabricated structures, for example in a photo, may appear different while still incorporating the claimed structures of the illustrated embodiments (e.g., walls may not be exactly orthogonal to one another in actual fabricated devices). Moreover, the drawings may only show the structures useful to understand the illustrated embodiments. Additional structures known in the art may not have been included to maintain the clarity of the drawings. For example, not every layer of a device is necessarily shown. “An embodiment”, “various embodiments” and the like indicate embodiment(s) so described may include particular features, structures, or characteristics, but not every embodiment necessarily includes the particular features, structures, or characteristics. Some embodiments may have some, all, or none of the features described for other embodiments. “First”, “second”, “third” and the like describe a common object and indicate different instances of like objects are being referred to. Such adjectives do not imply objects so described must be in a given sequence, either temporally, spatially, in ranking, or in any other manner. “Connected” may indicate elements are in direct physical or electrical contact with each other and “coupled” may indicate elements cooperate or interact with each other, but they may or may not be in direct physical or electrical contact. Phrases such as “comprising at least one of A or B” include situations with A, B, or A and B. Further, throughout this disclosure, “metallic surface” is used to refer collectively to all types of electrically conductive surfaces, including but not limited to carbon fiber surfaces.

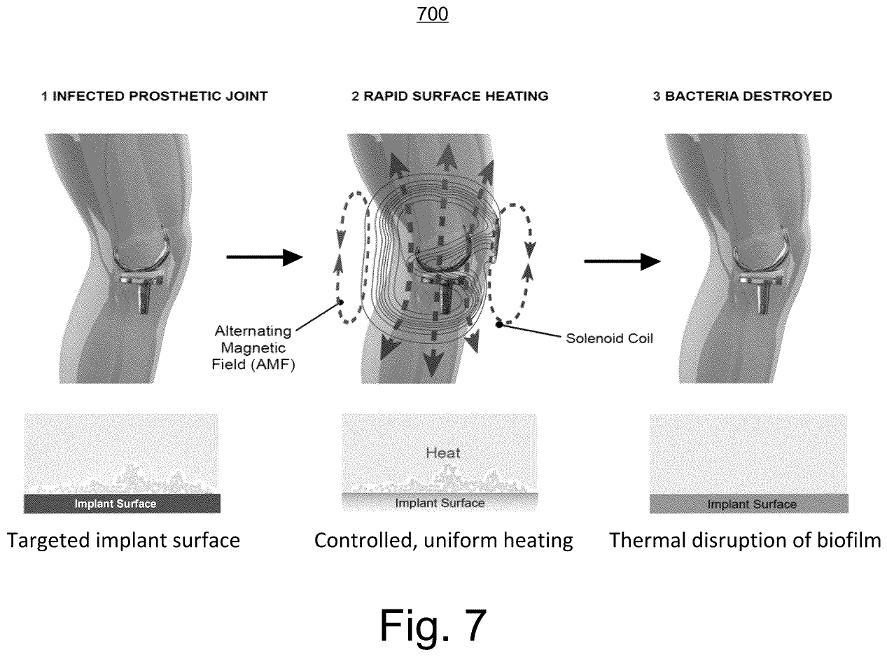

The present inventors have brought AMF treatment systems, devices, and technologies into practicality through a number of advances, improvements, and innovations. FIG. 7 illustrates 700 the process in general, using one or more embodiments of the present inventions. An infected prosthetic joint which has a biofilm infection on one or more target implant surfaces is subjected to a specifically-formed and shaped alternating magnetic field (AMF) using one or more solenoid coils to cause thermal disruption of the biofilm on the targeted implant surface.

Applicant has determined precise positioning of the AMF transducer coil(s) outside of the patient and relative to the target metal implant within the patient is important for accurate surface interaction (e.g., heating) of the implant. Applicant determined misalignment of the implant (i.e., incorrect positioning of the implant relative to the transducer coil(s) can result in under or over exposure of the implant, which could impact the safety and efficacy of the treatment. User error and patient-to-patient variability need to be mitigated to achieve treatment within a safe envelope of operation. Because the implant is not visible in the non-invasive AMF treatment, Applicant determined a method and system is needed to ensure repeatable and precise positioning of the coil or coils with respect to the implant.

Still further, Applicant determined that the performance of a simple RF magnetic field produced in a center or core of a cylindrical-shaped transducer could be significantly improved in efficacy of treating in vivo infections on surfaces of implants by shaping the field to conform it closely to the contour of the infected metallic surface, thereby minimizing energy into unrelated elements of the implant, and reducing trauma to nearby tissue.

Even further, Applicant determined that, upon shaping the field to correspond to the metallic surface to be treated, the energy level of the field could be increased significantly, thereby further increasing the efficiency of the non-intrusive AMF treatment machine and process.

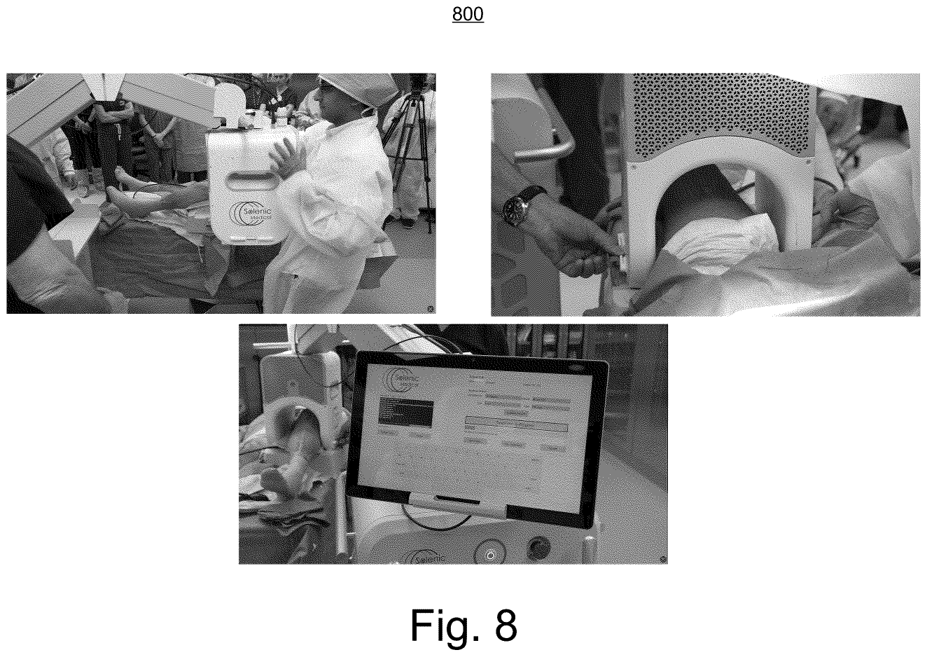

In at least one embodiment of the present invention, a knee prosthetic joint is treated by placing a transducer treatment unit over, around, under or besides the prosthetic joint while it is still implanted in the patient, as shown 800 in FIG. 8.

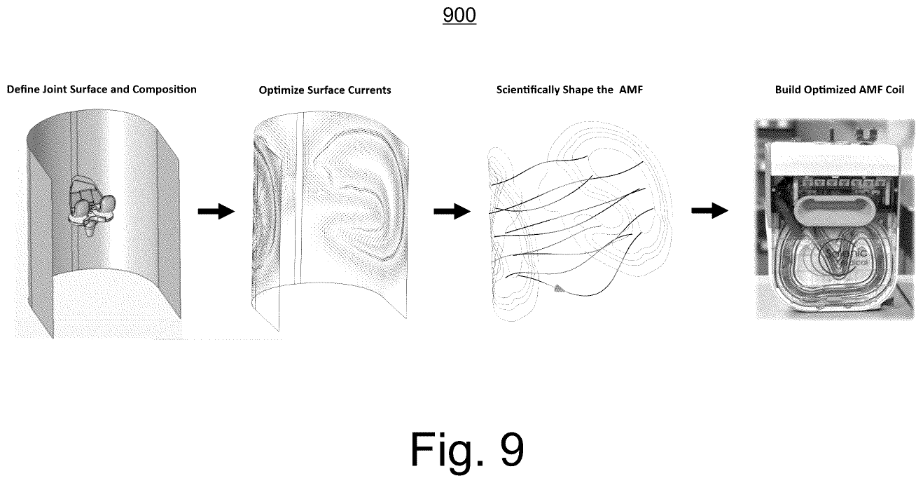

Transducers Specifically-Designed for Make, Model and Size of Implants. According to at least one embodiment of the present invention, each transducer is designed to produce a shaped density of magnetic field for optimal surface heating for a specific make, model, and size of an implant, such as a specific make, model and size of knee or hip or should prosthetic joint, or for a particular make, model and size of bone screw. FIG. 9 shows 900 a generalized process for designing each transducer, including:
a. defining the implant's target surface and composition;
b. optimizing surface currents, and thereby thermal interruption of biofilm growth, on the target surfaces of the specific make, model and size of implant;
c. designing one or more inducer coil patterns which scientifically shape and AMF around or near the implant to cause the induced surface currents, and
d. building and distributing the specific transducer unit for use with a base console.

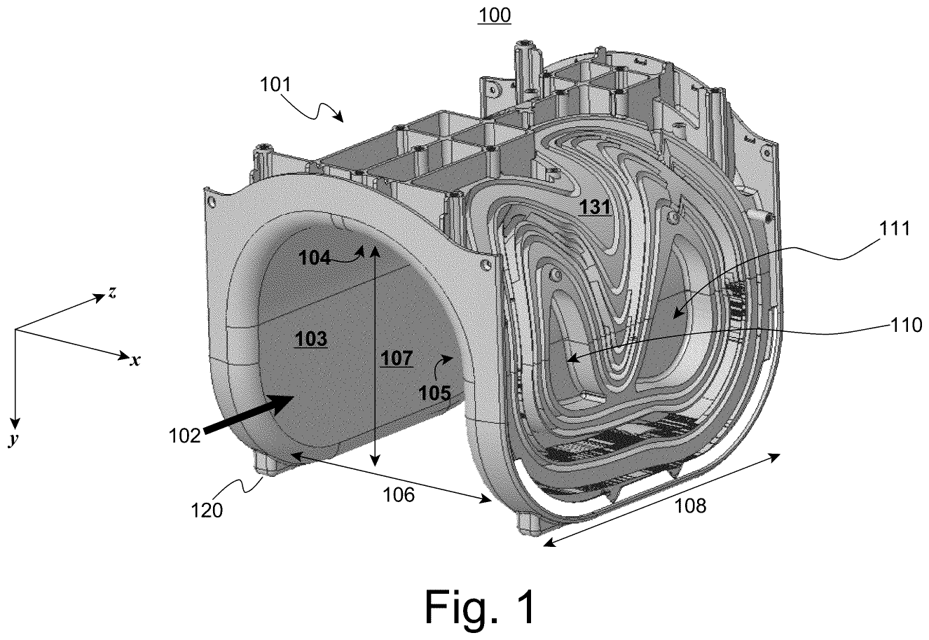

Turning now to FIG. 1, a perspective view 100 is shown of a particular example embodiment according to the related U.S. patent application Ser. No. 18/934,339 (hereinafter “the related invention”) invention for treating a knee replacement implant. Other treatment heads for other implant types, such as hips, shoulders, elbows, vertebrae, pins, plates and stents, are possible within the scope of the present invention, and may take different shapes and forms to generate appropriately shaped alternating magnetic fields. Therefore, the treatment head including magnetic field transducers will be referred to as a Treatment Transducer Coil Assembly (TTCA) throughout this document. Each TTCA for each different implant type to be treated will include a housing that acts as an enclosure for a plurality of windings, some other tank circuit components such as capacitors, temperature and current sensors, and other markings to assist with alignment of the TTCA around, near, above, below, in front of or behind the portion of the patient's body to be treated. Each TTCA will receive a high-power, high-voltage alternating electrical excitation signal from a signal generation section.

The related example embodiment of FIG. 1 has a lower enclosure section 101 with an upper cover (removed in this diagram). The enclosure sections are preferably constructed of a material that will not interfere with the alternating magnetic field, nor will it have currents induced into the enclosure portions. Certain plastics and resins are suitable for this sort of use, such as those already used in magnetic resonance imaging (MRI) housings. This related example, being for a knee joint implant, is arranged in an inverted U-shape, having two side panels 102, 105, and a top panel 104, and an open bottom so that it can be placed over a knee of a patient. The volume 102 defined between the side panels and top panel becomes a treatment chamber in which a certain shaped magnetic field is produced to provide for focused heating on the surface contours of the implant. This particular drop-down style is useful in a scenario where the patient is laying on a bed or table, so the knee does not need to be lifted in order to get proper positioning and alignment of the transducers around the implant. Other embodiments may have treatment head enclosures which allow for the limb or body portion to be slid axially into the enclosure, lowered down into the enclosure, placed in front of, behind, below, above or beside the enclosure as well. For certain implant treatments, the treatment head may only have one or two panels, although we will still refer to housing for the transducer coils as an “enclosure” (e.g., it encloses the coils but not necessarily encloses the body portion).

This particular related example embodiment has a foot feature 120 to allow it to rest stably on a surface, such as a treatment table or bed. For convenience, but not in a limiting sense, we will use three orthogonal axes x, y and z to assist in the understanding of the three-dimensional structures shown and disclosed. Those ordinarily skilled in the art will realize this is simply a convention chosen for reference, but does not limit the embodiments of the invention to particular orientation, whereas the produced alternating magnetic fields are not appreciably affected by gravity for treatment efficacy, so the inverted U-shaped structure would work effectively if used in a non-inverted U orientation or a vertical orientation, etc.

The width 106, height 107 and length 108 of the chamber 102 formed within the TTCA is sized to receive an appropriate body part, such as an adult knee or a child knee, which will contain the implant surface to be treated.

On the right-side panel 105 (right is for reference only from this viewing angle), an example transducer coil pattern formed by particularly shaped grooves and indentations is shown in which the lower enclosure section 101 produces two core areas or magnetic axes 110 and 111. This particular coil pattern, and its symmetrically mirrored pattern on the opposite side panel 103, takes a general shape of a B or even more accurately a German Eszett character β. The multiple windings around the two magnetic axes on each side panel increase the maximum possible magnetic field strength produced in the chamber 102. The particular shape of the windings allows for a specific contour of maximum magnetic field strength produced around the magnetic axes to focus maximum power into a virtual “surface” that nearly or closely matches the metallic surface of the implant to be treated. In this manner, the field is optimized to uniformly heat the entire implant conductive surface, whereas is may not be known where exactly a biofilm exist, and a minimum amount of heating of adjacent tissue is achieved. For other treatment transducers for other types of implants, the enclosure will hold the windings in other shapes and patterns, some of which may be asymmetrical, for the same, different or additional advantages and objectives.

Shown in FIG. 1 are a number of structural strengthening walls which assist the lower enclosure section 101 and its mating cover (not shown) to hold its shape, support the weight of the coils and other internal components, and in some embodiments, attach to a gantry or placement arm. The lower enclosure section 101 may be provided with a plurality of ports or holes to allow for fish wire or fish tape to be used during assembly to pull the transducer wire through the channels, grooves or indentations to achieve the intended coil shape. In other embodiments, the coils may be separately formed and placed as a unit into the treatment head enclosure. In still other embodiments, the transducer coil conductors may be of a printed circuit type or of a milled or shaped metal rods, with appropriate electric and thermal insulation applied where needed.

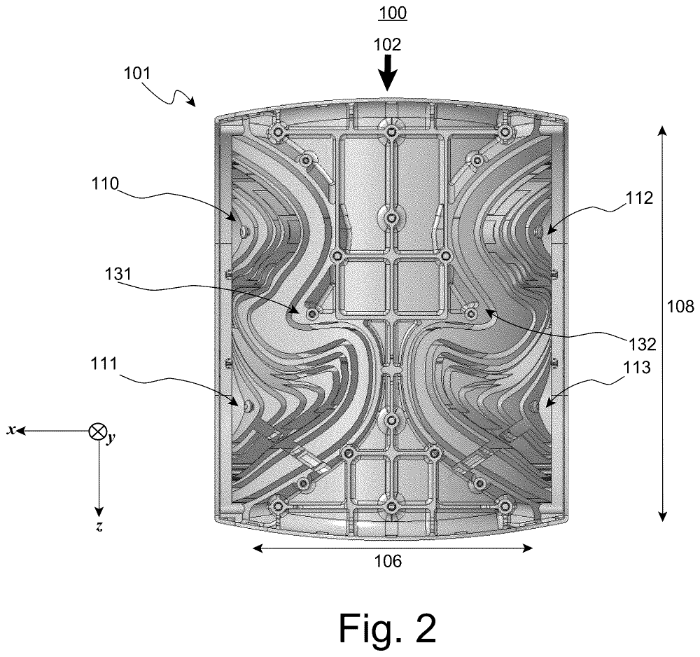

Referring now to FIG. 2, a top-down view of the related example embodiment of FIG. 1 is shown. From this view, one can see the symmetrically mirrored coil patterns and their magnetic axes 112 and 113 on the left side panel 103. In this non-perspective view, the y reference axis is pointing into the page as indicated by the circle with the X in it (e.g., as an arrow travels away from the viewer).

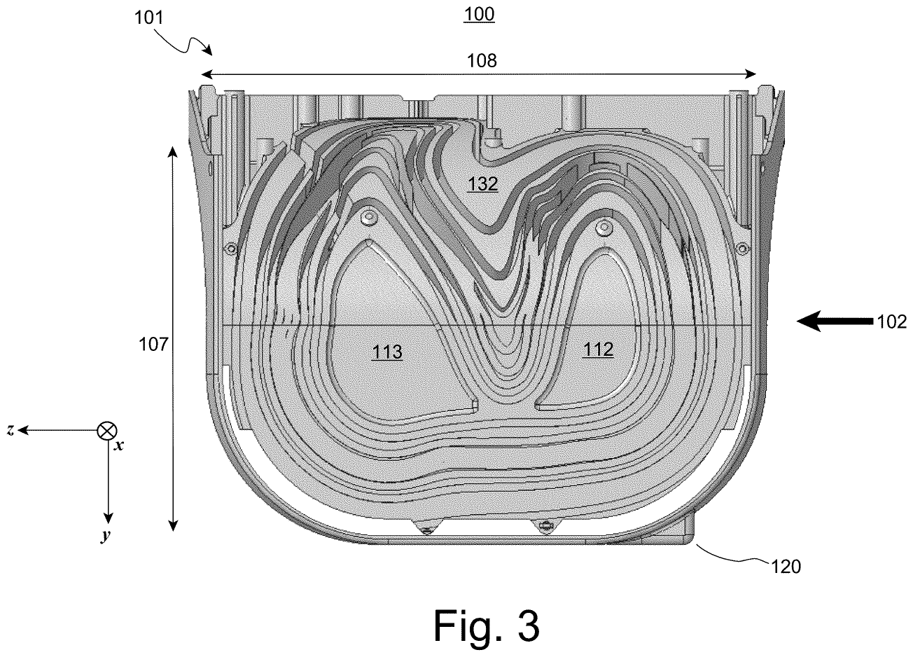

Referring now to FIG. 3, a side view of the left side 103 is shown in which the outer windings of the larger of the two loops wraps over the top of the lower enclosure section 101. In this non-perspective view, the x reference axis is pointing into the page. The foot feature 120 of this example embodiment is in clear view in this diagram as well.

In FIGS. 1, 2 and 3, the particular related example embodiment B-shaped coil pattern has a third magnetic axis formed at the intersection of the two loops wherein the conductors make a near 180-degree turn in the opposite direction of the loops, which we have labeled 131 and 132 in these diagrams. Depending on the size of the structures (winding shapes), frequencies involved, amplitudes of the signal, etc., these minor magnetic axes may be contributory to the shape of the contour of the maximum magnetic field produced in the treatment chamber.

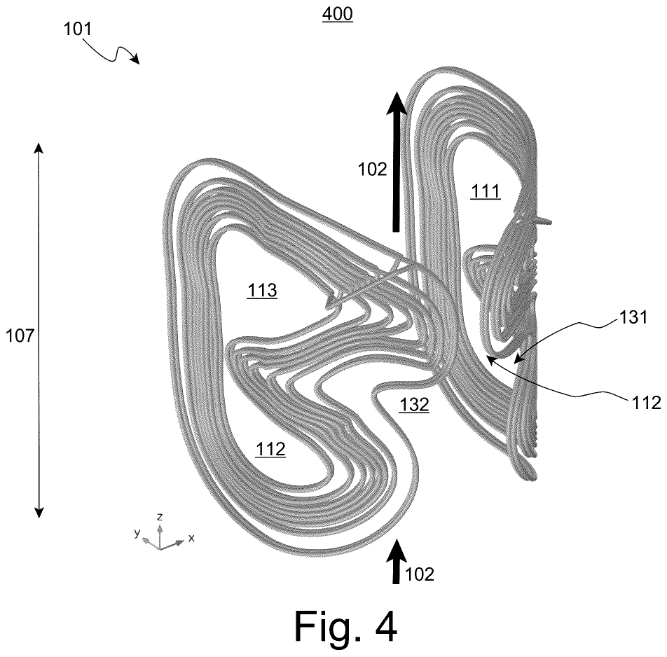

Turning to FIG. 4, an interconnection diagram 400 is shown for the conductors which are disposed in the channels, grooves or indentations of the side panels. This particular view shows the conductors in their three dimensionally-held arrangement without the structures that hold them in these shapes for convenience of the reader. Windings in this pattern are overlapped strategically to reduce electric potential between them. This helps reduce insulation, overall TTCA weight and space requirements, which enables extensive and easier maneuverability.

The foregoing related example coil-holding forms are preferably rigid and made of non-electrically conductive materials such as plastic, ABS, resin, and fiberglass. Other coil-holding forms may be of a flexible material, such as flexible PVC, vinyl, etc., such that the coils are held in the desired pattern to induce heating on the specific electrically-conductive implant surface, but the treatment device itself may be shaped around, draped, laid on, or wrapped around the portion of the patient's body in which the surface to be disinfected is implanted.

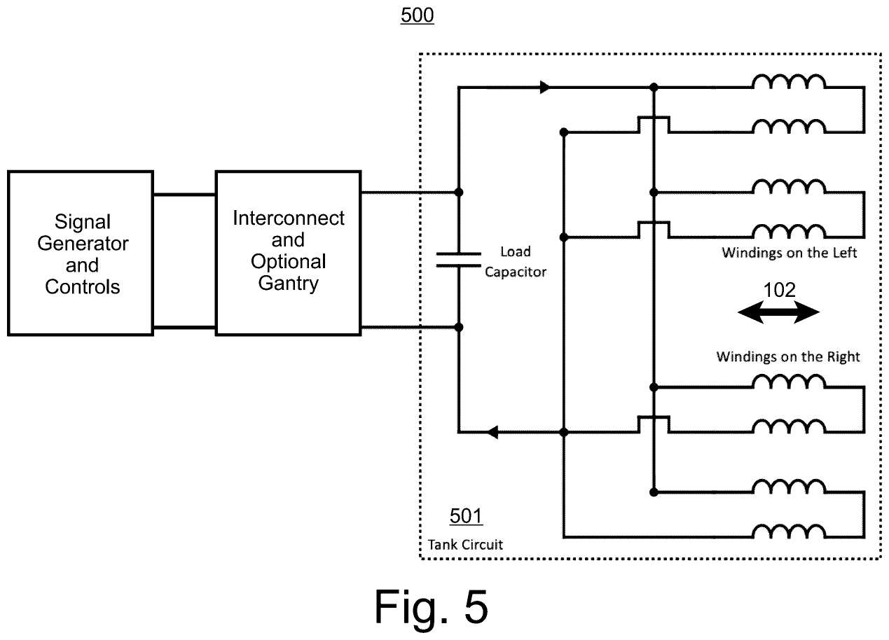

Transducers. The electronics of the transducers are included in the example embodiment 101, and in some embodiments, the mechanical positioning arm (“gantry”) may be considered to be part of this section as might be the mechanical components and elements for routing the transducer windings to produce the desired shaped field. Referring now to FIG. 5, a tank circuit 501 nominally includes the high-voltage, high-current in windings for creating and emitting the alternating magnetic field at the center of which an in vivo medical implant may be treated in the treatment chamber 102. The large inductive windings and one or more load capacitor(s) of the field generating and emitting portion of the example embodiment TTCA are shown in their schematic arrangement, into which an effective series resistance (ESR) is added when an implant for treatment is present in the chamber 102.

For various intended treatments, different embodiments may include different windings, with different induction values, and with different three dimensional arrangements of the windings to produce different field shapes, as previously stated. In at least one embodiment, fiber optic temperature sensors are placed in strategic locations within the transducer section in order to allow the controller section to monitor heating of the elements of the transducer section.

In the particular example embodiment, which is intended for treating a knee replacement, twelve (12) polyester film power capacitors model CSM 150/300 (0.17 μF each) available from Celem Passive Components Ltd. of Jerusalem, Israel, for the load capacitors.

In this particular example embodiment of the present invention, the circulating current in the TTCA during excitation and generation of the 216 kHz magnetic field is about 125 ARMS, with 14 nF of capacitance and 40 μH of inductance. Other values are possible for other TTCAs to treat other in vivo medical appliances such as hip replacements, elbow replacements, shoulder replacements, heart stents, peripheral artery stents, bone pins and fracture plates.

In at least one embodiment, the coils are fabricated using “Litz” (Litzendraht) wire, which is a bundled wire in which each strand is insulated from each other. These strands are twisted together in a way which equalizes impedance of all strands over a unit length, minimizing AC resistance. Each strand diameter is selected based on a target operating frequency. In one example embodiment according to the present invention, the TTCA contains coils using 1750 strands of 42 AWG Litz wire, such that the strand bundles an overall 8 AWG wire capable of carrying up to 50 A of current at 200 kHz.

In various embodiments:
a. the transducer coil pattern is configured such that they induce a current density over the entire surface of the implant as uniformly as possible, thereby uniformly increasing surface temperature of implant until it generates sufficient heat energy to disinfect the implant surface;
b. each different coil pattern corresponds to a contour of a specific surface to be disinfected, such as a specific make and model of implant;
c. irregularly shaped coil patterns with multiple loops are possible to reduce an average radius wherein each loop is not necessarily in the same pattern;
d. coil pattern can be continuous on the entire surface of the shape or it can be distributed over the shape with two or more not necessarily identical patterns; and
e. each coil pattern may be wound with a custom construction of Litz wire in either one or more layers.

Coupon Sensor. In at least one embodiment according to the related and present inventions, a “coupon” is a small piece of stainless steel, measuring about 20×20×3 mm which is placed in a convenient, fixed location within the TTCA, and to which one of the fiber-optic temperature sensors is affixed. The coupon will receive enough of the RF magnetic field strength to heat up enough during a treatment (ΔT about 30° C. after 90 seconds of an ˜1200 W maximum power treatment) to provide the controller section with another indirect measurement of RF magnetic field strength within the transducer coil assembly, with negligible effect on efficiency of TTCA to heat the implant.

Another Treatment Transducer Coil Assembly (TTCA). The foregoing TTCA and specific B-shaped coils were designed using the foregoing process for a specific make, model, and size of prosthetic knee implant. We now disclose a second TTCA coil design for a different but specific make, model and size of prosthetic knee implant.

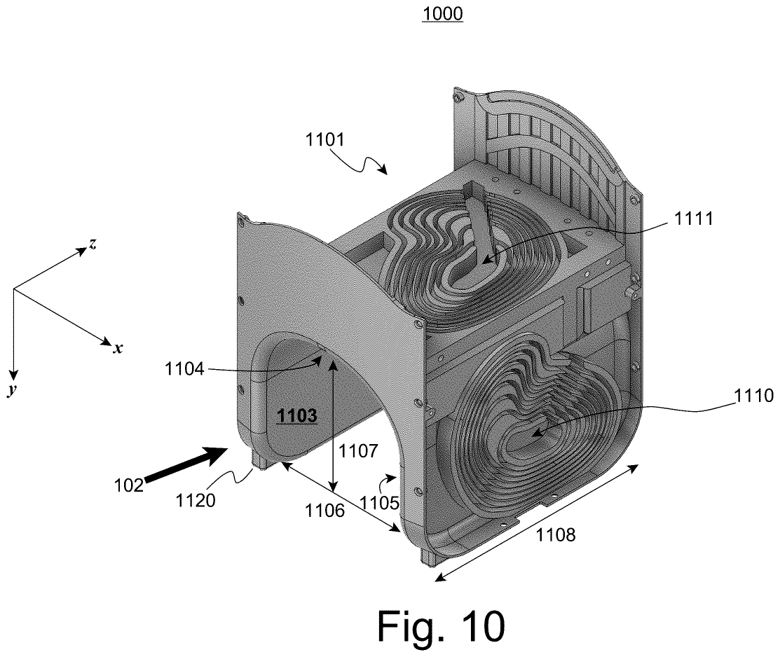

Referring now to FIG. 10, the second example embodiment, albeit for a different make, model and size of knee implant, has a lower enclosure section 1101 with an upper cover (removed in this diagram). As with the foregoing first kneel transducer, the enclosure sections are preferably constructed of a material that will not interfere with the alternating magnetic field, nor will it have currents induced into the enclosure portions. Certain plastics and resins are suitable for this sort of use, such as those already used in magnetic resonance imaging (MRI) housings. This related example is also arranged in an inverted U-shape, having two side panels 1103, 1105, and a top panel 1104, and an open bottom so that it can be placed over a knee of a patient. The volume 102 defined between the side panels and top panel becomes a treatment chamber in which a certain shaped magnetic field is produced to provide for focused heating on the surface contours of the implant.

This second example embodiment has similarly has a foot feature 1120 to allow it to rest stably on a surface, such as a treatment table or bed. For convenience, but not in a limiting sense, we will continue to use three orthogonal axes x, y and z to assist in the understanding of the three-dimensional structures shown and disclosed. Those ordinarily skilled in the art will realize this is simply a convention chosen for reference, but does not limit the embodiments of the invention to particular orientation, whereas the produced alternating magnetic fields are not appreciably affected by gravity for treatment efficacy, so the inverted U-shaped structure would work effectively if used in a non-inverted U orientation or a vertical orientation, etc.

The width 1106, height 1107 and length 1108 of the chamber 102 formed within the TTCA is sized to receive an appropriate body part, such as an adult knee or a child knee, which will contain the implant surface to be treated.

On the right-side panel 1105 and on the top panel 1104 (right and top are for reference only from this viewing angle), an second example transducer coil pattern formed by particularly shaped grooves and indentations is shown in which the lower enclosure section 1101 produces two core areas or magnetic axes 1110 and 1111. This particular coil pattern takes a general shape of a “bean” (oval with a dented side, a quartic-like curve). The multiple windings around the two magnetic axes on the top and side panels increase the maximum possible magnetic field strength produced in the chamber 102. The particular shape of the windings allows for a specific contour of maximum magnetic field strength produced around the magnetic axes to focus maximum power into a virtual “surface” that nearly or closely matches the metallic surface of the second specific implant to be treated. In this manner, the field is optimized to uniformly heat the entire implant conductive surface, whereas is may not be known where exactly a biofilm exist, and a minimum amount of heating of adjacent tissue is achieved. For other treatment transducers for other types of implants, the enclosure will hold the windings in other shapes and patterns, some of which may be asymmetrical, for the same, different or additional advantages and objectives.

Similarly to how they are shown in FIG. 1, a number of structural strengthening walls are shown 1000 in FIG. 10 which assist the lower enclosure section 1101 and its mating cover (not shown) to hold its shape, support the weight of the coils and other internal components, and in some embodiments, attach to a gantry or placement arm. The lower enclosure section 1101 may be provided with a plurality of ports or holes to allow for fish wire or fish tape to be used during assembly to pull the transducer wire through the channels, grooves or indentations to achieve the intended coil shape. In other embodiments, the coils may be separately formed and placed as a unit into the treatment head enclosure. In still other embodiments, the transducer coil conductors may be of a printed circuit type or of a milled or shaped metal rods, with appropriate electric and thermal insulation applied where needed.

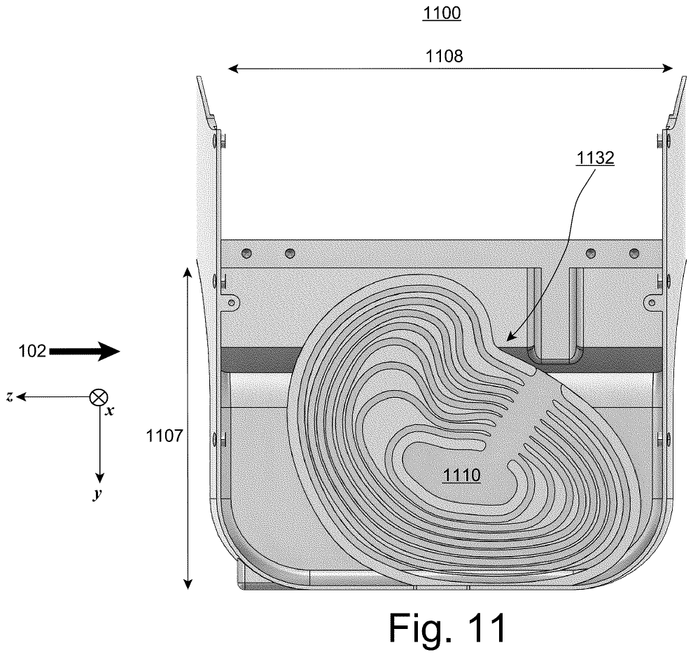

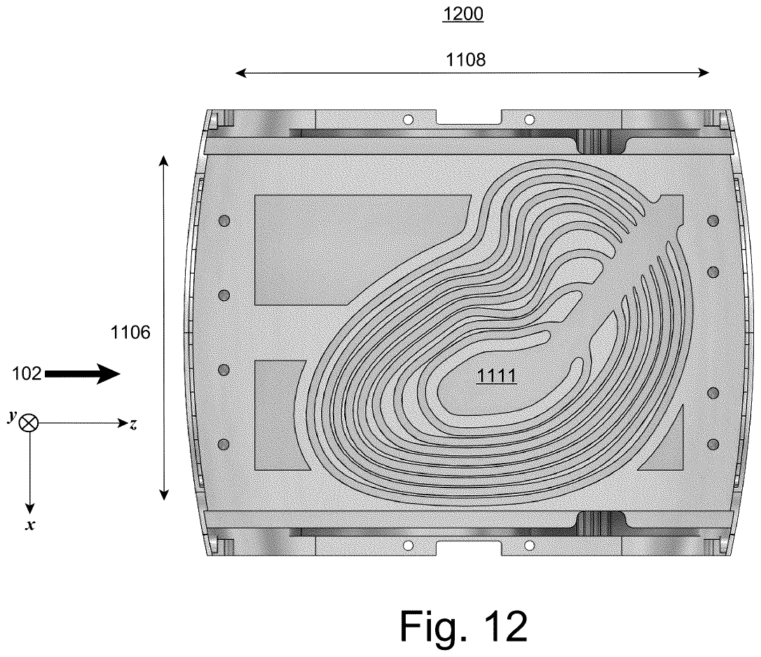

FIG. 11 shows the coil pattern grooves and indentations from a view of the right, which allows the approximate bean-shaped pattern to be viewed straight-on. FIG. 12 shows the coil pattern grooves and indentations from a top-down view, which allows the approximate bean-shaped pattern to be viewed straight-on. In some embodiments, there may be a third coil pattern formed on the other side of the TTCA.

As in the first coil embodiment, in at least one second embodiment, the coils are fabricated using “Litz” (Litzendraht) wire, which is a bundled wire in which each strand is insulated from each other. These strands are twisted together in a way which equalizes impedance of all strands over a unit length, minimizing AC resistance. Each strand diameter is selected based on a target operating frequency. In one example embodiment according to the present invention, the TTCA contains coils using 1750 strands of 42 AWG Litz wire, such that the strand bundles an overall 8 AWG wire capable of carrying up to 50 A of current at 200 kHz.

In various embodiments of this second coil pattern:
a. the transducer coil pattern is configured such that they induce a current density over the entire surface of the implant as uniformly as possible, thereby uniformly increasing surface temperature of implant until it generates sufficient heat energy to disinfect the implant surface;
b. each different coil pattern corresponds to a contour of a specific surface to be disinfected, such as a specific make and model of implant;
c. irregularly shaped coil patterns with multiple loops are possible to reduce an average radius wherein each loop is not necessarily in the same pattern;
d. coil pattern can be continuous on the entire surface of the shape or it can be distributed over the shape with two or more not necessarily identical patterns; and
e. each coil pattern may be wound with a custom construction of Litz wire in either one or more layers.

Controller Signals and Excitation Signal Reception. Each treatment head according to the related and present inventions is designed to receive a particular signal or range of signals according to frequency, amplitude and current specifications in order to treat a particular type of medical implant with alternating magnetic fields. A signal generating section provides a sufficiently-high power alternating electronic signal through an interconnect section to the shaped-field transducer of the foregoing designs. A controller section receives inputs from monitoring sensors, measuring sensors, and user interfaces, and generates control signals to various components and subsystems to accomplish the advanced functionality as described herein.

Interconnect Section. Because the TTCA must be co-located with the patient but the signal generation and control sections may be located several feet from the TTCA, an interconnect section of the example embodiment performs an important function of conveying the high-power, high-voltage signal from the signal generation to the TTCA. Each TTCA may be mounted on a positioning arm which will assist with the alignment and holding of the transducer section in place during treatments, and will hold the TTCA conveniently away from the treatment area between treatments to allow the patient and system operator freedom of movement in the area.

As such, the interconnect section can, in some embodiments, include the wires and conductors that run between the output of the tuning section to the beginning of any positioning arm, if present, and may also include the wires and conductors that pass alongside or within the positioning arm. In other embodiments, the wires and cables passing alongside or within the positioning arm may be considered part of the TTCA or even part of the arm itself (if the arm is categorized as a separate section of its own). Those skilled in the relevant arts will readily recognize that inclusion of such functions in one section or another is within ordinary design options, and all such embodiment variations are within the scope and spirit of the present invention.

Cables and wires should be designed in conventional manners for safety, EMI/EMC requirements, trip hazards, overhead plenum air return requirements, and pinch and wear protection. In at least one embodiment, the impedance of the interconnect section is designed to present a 50 Ω impedance to either end of it for the high-power, high-voltage conductors. In other embodiments, particularly for treatment of smaller implants, the interconnect section may be minimized or even eliminated in favor of having the signal generation and controls within the same unit as the transducers, with proper magnetic shielding as appropriate.

Positioning (Gantry) Arm. It is expected that some treatment heads may be heavy and/or clumsy to move manually, and alignment of the treatment head with the in vivo implant to be treated may be greatly assisted by a gantry or positioning and placement arm. Such an arm could use a variety of weight offsetting mechanisms, such as springs, counterweights, cables, and even motors, to reduce the strength needed of a technician to move the treatment head into place for treatment and to move it into a stowing position between treatments. Such a gantry can also be used to route and hold the wires and fiber optic conductors of the interconnect section of the system, thereby producing a medical appliance user-experience similar to that of an x-ray machine or an overhead light.

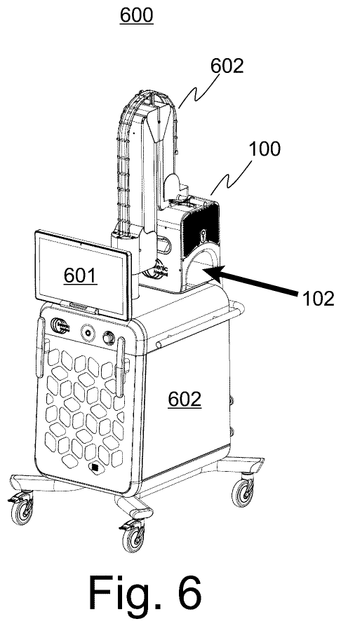

System as a Whole. An example embodiment of a treatment system for applying alternating magnetic fields to in vivo medical implants via a treatment head or TTCA of the present invention is shown 600 in FIG. 6, in which a control system with a user interface 601 and a main unit 602 housing the excitation signal generation circuits provides the excitation signal via an interconnect 602 through a gantry to the inventive TTCA 100. Other system embodiments which interface to, utilize, or leverage the present invention are possible within the scope of the present invention.

Conclusion. The foregoing description of the embodiments of the invention has been presented for the purposes of illustration and description. It is not intended to be exhaustive or to limit the invention to the precise forms disclosed. This description includes terms, such as left, right, top, bottom, over, under, upper, lower, first, second, etc. that are used for descriptive purposes only and are not to be construed as limiting. For example, terms designating relative vertical position refer to a situation where a side of a substrate is the “top” surface of that substrate; the substrate may actually be in any orientation so that a “top” side of a substrate may be lower than the “bottom” side in a standard terrestrial frame of reference and still fall within the meaning of the term “top.” The term “on” as used herein does not indicate that a first layer “on” a second layer is directly on and in immediate contact with the second layer unless such is specifically stated; there may be a third layer or other structure between the first layer and the second layer on the first layer. The embodiments of a device or article described herein can be manufactured, used, or shipped in a number of positions and orientations. Persons skilled in the relevant art can appreciate that many modifications and variations are possible in light of the above teaching. Persons skilled in the art will recognize various equivalent combinations and substitutions for various components shown in the Figures. It is therefore intended that the scope of the invention be limited not by this detailed description, but rather by the claims appended hereto.

I/We claim:

1. An alternating magnetic field transducer for in vivo disinfection of electrically-conductive surfaces of medical devices, parts and components implanted internally into a patient body portion, comprising:
a coil-holding form for positioning adjacent to a patient body portion having a electrically-conductive surface to be disinfected; and
one or more coils physically held in a physical pattern and supported by the form, which, when excited by an excitation signal, generates a shaped electromagnetic field and provides focused induced heating of the electrically-conductive surface to be disinfected;
thereby providing selective in vivo heating of the electrically-conductive surface and reducing heating of adjacent patient tissue.

2. The alternating magnetic field transducer as set forth in claim 1 wherein the physical pattern comprises a first pattern having a general “bean” shape, and a second pattern having a general “bean” shape, wherein the first and second bean shapes may be different, thereby producing, when excited by an excitation signal, the shaped electromagnetic field to match a specific contour of the specific surface to be disinfected.

3. The alternating magnetic field transducer as set forth in claim 1 wherein the coil-holding form comprises a U-shape structure defining a volume within the U-shape for receiving the patient body portion.

4. The alternating magnetic field transducer as set forth in claim 1 wherein the coil-holding form is rigid.

5. The alternating magnetic field transducer as set forth in claim 1 wherein the coil-holding form is flexible.

6. The alternating magnetic field transducer as set forth in claim 1 wherein the coil-holding form defines a volume to receive and at least partially envelop a patient body portion having the surface to be disinfected.

7. The alternating magnetic field transducer as set forth in claim 1 wherein the coil-holding form comprises a plurality of shaped grooves, indentations, posts, or a combination thereof into which, through which, and around which coil conductors are received to form the coil pattern.

8. The alternating magnetic field transducer as set forth in claim 1 wherein one or more coils comprises Litz wire.

9. The alternating magnetic field transducer as set forth in claim 1 wherein one or more coils comprises a printed circuit board.

10. The alternating magnetic field transducer as set forth in claim 1 wherein one or more coils is formed separately from the holding enclosure structure.

11. The alternating magnetic field transducer as set forth in claim 1 wherein in a physical shape of the coil-holding form is adapted for positioning relative to the patient body portion in a position selected from the group consisting of over, under, around, in front of, behind, below, above, below and beside the patient body portion having the surface to be disinfected.

12. The alternating magnetic field transducer as set forth in claim 11 wherein the positioning comprises one or more movements selected from the group consisting of sliding axially, lowering down, raising up, closing around, wrapping around, laying upon and horizontally moving.

13. The alternating magnetic field transducer as set forth in claim 1 further comprising a gantry arm supporting the coil-holding form.

14. The alternating magnetic field transducer as set forth in claim 1 further comprising one or more tank circuit components in electrical communication with the one or more coils.

15. The alternating magnetic field transducer as set forth in claim 14 wherein the one or more tank circuit components comprise one or more components selected from the group of a capacitor, a temperature sensor, and a current sensor.

16. The alternating magnetic field transducer as set forth in claim 1 further comprising a coupon sensor disposed near the one or more coils to be heated by receiving a portion of the alternating magnetic field produced by the coils when excited by an excitation signal, and a temperature sensor disposed on or near the coupon sensor for measuring the heating effect.
